(19) United States
(12) Patent Application Publication
(10) Pub. No.: US 2006/0074248 A1
(43) Pub. Date: Apr. 6, 2006

(54) Process for the stereoselective synthesis of lactones

(76) Inventor: Christof Wehrli, Witterswil (CH)

Correspondence Address: NIXON & VANDERHYE, PC, 901 NORTH GLEBE ROAD, 11TH FLOOR, ARLINGTON, VA, 22203, US

(21) Appl. No.: 10/543,719

(22) PCT Filed: Apr. 20, 2004
(86) PCT No.: PCT/EP04/04172

§ 371 Date: Jul. 28, 2005

(30) Foreign Application Priority Data
Apr. 22, 2003 (EP) 03009198.7

(51) Int. Cl. C07D 491/02

(52) U.S. Cl. 548/303.1

(57) ABSTRACT

A process is described for the stereoselective synthesis of a chiral lactone which can be used as an intermediate in the synthesis of biotin. The process includes a reaction sequence in which a cyclic meso-carboxylic acid anhydride is converted with the aid of a chiral alcohol with ring opening into a dicarboxylic acid monoester. With respect to the dicarboxylic acid monoester obtained from the cyclic meso-carboxylic acid anhydride and the chiral alcohol, the reaction proceeds diastereoselectively. The reaction is performed in the presence of a specific catalyst improving the diastereomeric purity of the dicarboxylic acid monoester.

The present invention is concerned with a process for the stereoselective synthesis of a chiral lactone of the general formula (I) 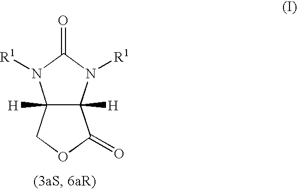
wherein R¹ is benzyl, α-phenylethyl, allyl, 1-furyl, 2-furyl, 1-thienyl, 2-thienyl or p-methoxy-benzyl. The process embraces the diastereoselective esterification of a cyclic carboxylic acid anhydride with a chiral alcohol in the presence of a particular catalyst.

The present invention is concerned with a reaction sequence in which a cyclic meso-carboxylic acid anhydride is converted with the aid of a chiral alcohol with ring opening into a dicarboxylic acid monoester. With respect to the dicarboxylic acid monoester obtained from the cyclic meso-carboxylic acid anhydride and the chiral alcohol, the reaction proceeds diastereoselectively. In a preferred embodiment of the process in accordance with the invention the ester group of the dicarboxylic acid monoester is selectively reduced in a further step of the reaction sequence to give a hydroxycarboxylic acid which recyclizes to a lactone. With respect to the lactone obtained from the cyclic meso-carboxylic acid anhydride the reaction sequence proceeds entirely enantioselectively.

The enantioselective synthesis of lactones from cyclic meso-carboxylic acid anhydrides with the aid of chiral alcohols is known in the state of the art.

EP-A 161 580 discloses a process for the manufacture of a chiral lactone which can be used as an intermediate in the synthesis of biotin. In a first reaction step cis-1,3-dibenzyl-hexahydro-1H-furo[3,4-d]imidazole-2,4,6-trione is reacted with a particular secondary chiral alcohol in the presence of a catalyst. Tertiary amines such as, for example, diazabicyclooctane (DABCO), diazabicycloundecene (DBU), p-dimethylaminopyridine or also trialkylamines containing lower alkyl residues, such as triethylamine, are mentioned as suitable catalysts. Subsequent to this, the selective reduction of the ester group of the formed dicarboxylic acid monoester is effected with the aid of a complex borohydride, the hydroxycarboxylic acid formed recyclizing to a lactone. The lactone is thereby obtained as the crude product in optical purities of 61.3 to 95.8% ee depending on the chosen reaction conditions.

An optical purity of, for example, 95% signifies that in addition to 97.5% of the desired enantiomer 2.5% of the undesired enantiomer is also present. As a consequence of this impurity of the end product the process disclosed in EP-A 161 580 has the disadvantage that the yield of the desired reaction product is reduced on the one hand by the content of the undesired enantiomer and on the other hand the reaction product cannot, however, be used in the further synthesis without prior complicated purification (recrystallization, enantiomer separation, etc). In the case of a multi-stage enantioselective synthesis any additional operational step is associated with considerable costs, not least since in practice it always results in a further decrease in the overall yield.

A further disadvantage of the process disclosed in EP-A 161 580 is that the catalysts disclosed there, such as e.g. trimethylamine or DABCO, are soluble in water, so that their recovery is possible only by separation procedures which are complicated and cost-intensive.

There therefore exists a need for a process for the enantioselective synthesis of lactones from cyclic carboxylic acid anhydrides which has advantages over the processes of the state of the art.

This goal is achieved by the subject matter of the patent claims.

It has surprisingly been found that the diastereoselectivity in a process for the esterification of a cyclic carboxylic acid anhydride of the general formula (II) 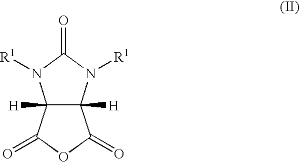
wherein R¹ is benzyl, α-phenylethyl, allyl, 1-furyl, 2-furyl, 1-thienyl, 2-thienyl or p-methoxy-benzyl, with a chiral alcohol of the general formula (III) 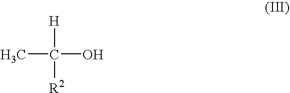
wherein R² is a residue of the general formulae (IV a-f) 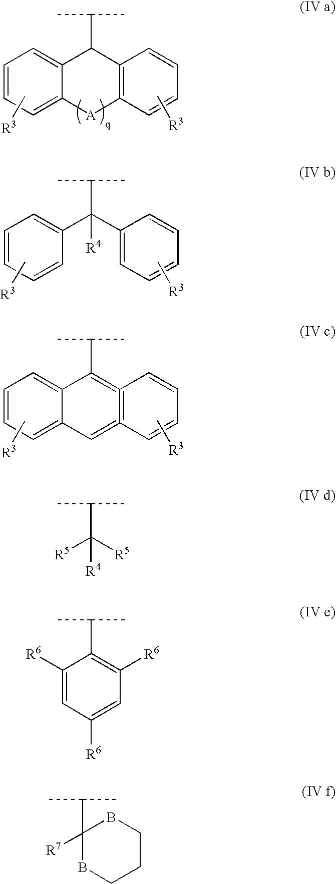
wherein
R³ is hydrogen, fluorine, chlorine, bromine, iodine, C₁-C₆-alkyl or C₁-C₆-alkoxy,
R⁴ is hydrogen, hydroxyl, C₁-C₆-alkyl, C₁-C₆-alkoxy or phenyl,
R⁵ is C₃-C₇-cycloalkyl, phenyl optionally substituted with chlorine or methyl, pyridyl, pyrrolyl, thienyl or furyl,
R⁶ is hydrogen or C₁-C₆-alkyl,
R⁷ is C₁-C₆-alkyl or phenyl,
A is sulphur or a methylene group, q being the integer 1 when A is sulphur or q being the integer 1 or 2 when A is a methylene group, and

B is sulphur, —SO₂— or a methylene group,

can be improved when the process includes the step

(a) bringing together the cyclic carboxylic acid anhydride with the chiral alcohol in the presence of a catalyst of the general formula (V) 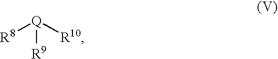
wherein
Q is nitrogen or phosphorus and
R⁸, R⁹ and R¹⁰ are each independently
(i) C₂-C₁₈-alkyl in which optionally up to two methylene groups can be replaced by oxygen, or
(ii) phenyl-C₁-C₄-alkyl in which optionally one methylene group can be replaced by oxygen, or
(iii) phenyl,
with the proviso that
when one of the residues R⁸, R⁹ and R¹⁰ is phenyl the other two residues are not phenyl and
when R⁸, R⁹ and R¹⁰ are each C₂-C₁₈-alkyl at least one of the three substituents R⁸, R⁹ or R¹⁰ comprises at least 3 carbon atoms.

In the present description the term “alkyl”, whether as such or as part of “alkoxy”, can be linear or branched.

In a preferred embodiment the radicals R⁸, R⁹ and R¹⁰, each independently, have the following significances: C₃-C₁₆-alkyl, more preferably propyl, such as n-propyl or iso-propyl; butyl, such as n-butyl, sec-butyl, iso-butyl or tert-butyl; pentyl, such as n-pentyl or neo-pentyl; hexyl; heptyl; octyl; nonyl; decyl; undecyl; dodecyl; tridecyl; tetradecyl; pentadecyl or hexadecyl.

In a preferred embodiment of the present invention in the radicals R⁸, R⁹ and R¹⁰ up to two methylene groups are independently replaced by oxygen. Preferably, however, the methylene groups directly linked to the residue Q are not replaced by oxygen. Furthermore, the terminal methylene group is preferably also not replaced by an oxygen atom. When two methylene groups are replaced by oxygen, the two oxygen atoms within the radical are preferably spaced from each other by at least one methylene group.

When R⁸, R⁹ or R¹⁰ is phenyl-C₁-C₄-alkyl in which one methylene group is replaced by oxygen, the group can be for example phenyl-C₁-C₃-alkoxy.

In the reaction of the cyclic carboxylic acid anhydride with the chiral alcohol there result basically two diastereomeric dicarboxylic acid monoesters of the general formulae (VI) and (VII), which can be present in the form of the free carboxylic acids or as their salts:

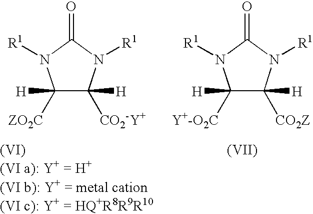

In the dicarboxylic acid monoesters of the general formulae (VI) and (VII) R¹ has the above significance, Z is the esterified form of the chiral alcohol of the general formula (III), i.e. the appropriate group of the general formula R²CH(CH₃)—, and Y⁺, depending on the reaction procedure which is chosen, is either a proton, a metal cation or the protonated, quaternary cation (HQ⁺R⁸R⁹R¹⁰) of the catalyst of the general formula (V).

When Y⁺ in the dicarboxylic acid monoester of the general formula (VI) is a proton, then the “dicarboxylic acid monoester” is a dicarboxylic acid monoester of the general formula (VI a); when Y⁺ is a metal cation then the “dicarboxylic acid monoester” is the appropriate metal salt of the dicarboxylic acid monoester, of the general formula (VI b); and when Y⁺ is the protonated, quaternary cation of the catalyst of the general formula (V) then the “dicarboxylic acid monoester” is the appropriate quaternary ammonium or phosphonium salt of the dicarboxylic acid monoester (hereafter referred to as quaternary ammonium or phosphonium dicarboxylic acid monoester), of the general formula (VI c).

By the use of a catalyst of the general formula (V) the dicarboxylic acid monoester of the general formula (VI) is obtained in improved diastereomeric purity over that of the dicarboxylic acid monoester of the general formula (VII).

In a preferred embodiment of the process in accordance with the invention the substituents R¹ in the cyclic carboxylic acid anhydride of the general formula (II) is benzyl, i.e. the cyclic carboxylic acid anhydride of the general formula (II) is preferably cis-1,3-dibenzylhexahydro-1H-furo[3,4-d]imidazole-2,4,6-trione.

Chiral alcohols of the general formula (III) are known in the state of the art. Reference can be made, for example, to EP-A-161 580. In a preferred embodiment of the process in accordance with the invention the substituent R² in the chiral alcohol of the general formula (III) is a residue of the general formula (IV d). Moreover, it is especially preferred that in the residue of the general formula (IV d) R⁴ is hydrogen or hydroxyl and R⁵ is phenyl optionally substituted with chlorine or methyl, or is thienyl or 2-furyl. In an especially preferred embodiment of the process in accordance with the invention the chiral alcohol is (S)-1,1-diphenyl-1,2-propanediol. In accordance with the invention the enantiomeric purity of the chiral alcohol is preferably at least 90% ee, more preferably at least 98% ee, especially at least 99% ee.

The substituents R⁸, R⁹ and R¹⁰ in the catalyst of the general formula (V) are each independently C₃-C₁₂-alkyl in a preferred embodiment of the process in accordance with the invention. In a more preferred embodiment of the process in accordance with the invention the catalyst of the general formula (V) is selected from the group consisting of tripropylamine, tributylamine, tripentylamine, trihexylamine, triheptylamine, trioctylamine, tridodecylamine and tributylphosphine, the catalyst especially preferably being selected from the group consisting of tributylamine, trihexylamine, trioctylamine; tridodecylamine and tributylphosphine.

The use of catalysts of the general formula (V), especially the use of tributylamine, trihexylamine; trioctylamine, tridodecylamine or tributylphosphine, has, in addition to the improved diastereoselectivity of the esterification, the further advantage that the catalysts are practically insoluble in water, which simplifies their recovery and thus reduces the overall costs of the process.

The reaction of the cyclic carboxylic acid anhydride with the chiral alcohol in the presence of the catalyst is preferably effected under an inert gas atmosphere, for example under nitrogen or argon, preferably at temperatures of −50° C. to +60° C., particularly at temperatures of −20° C. to +20° C.

In a preferred embodiment of the process in accordance with the invention 0.8 to 1.2 mol equivalents, preferably 0.9 to 1.1 mol equivalents, especially 1 mol equivalent, of the cyclic carboxylic acid anhydride are used based on the chiral alcohol.

In the process in accordance with the invention preferably 0.01 to 1.5 mol equivalents, especially either 0.95 to 1.05 mol equivalents or catalytic amounts of 0.05 to 0.2 mol equivalents, of the catalyst are used based on the cyclic carboxylic acid anhydride used.

The reaction of the cyclic carboxylic acid anhydride with the chiral alcohol is preferably effected in an inert, anhydrous, organic solvent. As solvents there can be named especially aromatic hydrocarbons such as benzene, toluene, xylene, anisole and chlorobenzene; ethers such as diethyl ether, tetrahydrofuran and dioxan; polyethers such as monoglyme, diglyme and triglyme; hydrocarbons such as pentane, hexane, heptane, cyclohexane and petroleum ether; halogenated hydrocarbons such as methylene chloride and chloroform; or also dimethyl-formamide, dimethyl sulphoxide, acetonitrile or carbon disulphide. Non-polar solvents, preferably aromatic hydrocarbons, especially toluene, are preferred. Non-polar solvents such as toluene have the advantage that, depending on the reaction conditions, the resulting dicarboxylic acid monoesters of the general formula (VI) can precipitate as the free dicarboxylic acid monoesters of the general formula (VI a), as a result of which they can be isolated with high diastereomeric purity, optionally as an intermediate step.

The dicarboxylic acid monoester of the general formula (VI) obtained by the esterification of the cyclic carboxylic acid anhydride with the chiral alcohol in the course of the process in accordance with the invention can be re-converted into the lactone of the general formula (I). For this purpose in accordance with the invention the ester group of the dicarboxylic acid monoester is reduced with a suitable selective reducing agent to give a hydroxycarboxylic acid which can be cyclized to the lactone. The selective reduction of the ester group of the dicarboxylic acid monoester can be carried out immediately after the esterification, but it is also possible to first isolate the obtained dicarboxylic acid monoester before it is reacted with the selective reducing agent. The isolation can be effected after formation of the quaternary ammonium or phosphonium salt with the catalyst of the general formula (V) [quaternary ammonium or phosphonium dicarboxylic acid monoester of the general formula (VI c)], after formation of the free acid [dicarboxylic acid monoester of the general formula (VI a)] or, however, preferably after conversion into a metal salt [dicarboxylic acid monoester metal salt of the general formula (VI b)].

In a preferred embodiment the process in accordance with the invention includes in addition to step (a), whether leading to the free acid or to the quaternary ammonium or phosphonium dicarboxylic acid monoester of the general formula (VI c), the step

(b) conversion of this form of the dicarboxylic acid monoester obtained in step (a) into a metal salt of the general formula (VI b).

In accordance with this embodiment of the invention the free acid or quaternary ammonium or phosphonium dicarboxylic acid monoester of the general formula (VI a) or (VI c) is preferably converted into an alkali metal salt, more preferably into the lithium salt, the following dicarboxylic acid monoester metal salt of the general formula (VI b) being thereby obtained: 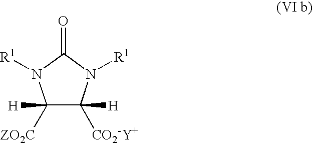
wherein R¹ is benzyl, α-phenylethyl, allyl, 1-furyl, 2-furyl, 1-thienyl, 2-thienyl or p-methoxy-benzyl, Z is the esterified form of the chiral alcohol of the general formula (III), i.e. the appropriate group of the general formula R²CH(CH₃)—, and Y⁺ is an alkali metal cation, preferably Li⁺.

In a preferred embodiment of the process in accordance with the invention cis-1,3-dibenzyl-hexahydro-1H-furo[3,4-d]imidazole-2,4,6-trione is reacted in step (a) with (S)-1,1-diphenyl-1,2-propanediol and the obtained dicarboxylic acid monoester, be it the free acid or its quaternary ammonium or phosphonium salt, is converted into the lithium salt of the following formula:

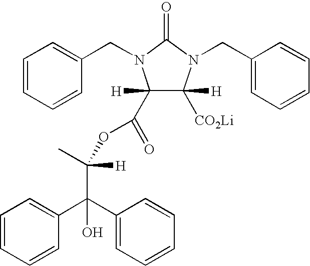

It has surprisingly been found that the conversion of the dicarboxylic acid monoester, be it the free acid or its quaternary ammonium or phosphonium salt, into a metal salt has the advantage that the resulting dicarboxylic acid monoester metal salt can be precipitated in improved diastereomeric purity. The diastereomeric purity of the precipitate is thereby higher than the diastereomeric purity of the reaction product which is obtained immediately from the reaction of the cyclic carboxylic acid anhydride with the chiral alcohol and which, depending on the reaction conditions, can be present in dissolved or suspended form, whereby in this case the free carboxylic acid function of the dicarboxylic acid monoester can be present wholly or partly in the form of the protonated, quaternary ammonium or phosphonium salt form of the catalyst of the general formula (V). Should the dicarboxylic acid monoester be precipitated as the metal salt, then non-polar solvents which are practically immiscible or only very slightly miscible with water are preferred, toluene being especially preferred.

The conversion of the dicarboxylic acid monoester, be it the free acid or its quaternary ammonium or phosphonium salt, into a metal salt can be effected, for example, by bringing together the dicarboxylic acid monoester, be it the free acid or its quaternary ammonium or phosphonium salt, with the corresponding metal hydroxide. Suitable metal hydroxides are, for example, lithium hydroxide, sodium hydroxide, potassium hydroxide, magnesium hydroxide and calcium hydroxide, of which lithium hydroxide is especially preferred.

The reaction temperature is preferably 10° C. to 60° C., more preferably 20° C. to 50° C. Preferably 1 to 2, more preferably 1 to 1.5, especially 1.01 to 1.3, mol equivalents of the metal hydroxide are added based on the dicarboxylic acid monoester. The metal hydroxide is preferably added in aqueous solution, the concentration of the aqueous solution preferably being 0.1 to 4 mol/l, more preferably 0.3 to 1.5 mol/l.

In a preferred embodiment of the process in accordance with the invention the process includes, in addition to step (a) or in addition to steps (a) and (b), the step

(c) bringing together the dicarboxylic acid monoester, be it the free acid or its quaternary ammonium or phosphonium salt, obtained in step (a) or the dicarboxylic acid monoester metal salt obtained in step (b) with a reducing agent which is selective for ester groups.

Reducing agents which selectively reduce ester groups but not the free carboxyl group or its metal or quaternary ammonium or phosphonium salt form are known to the person skilled in the art. Reference can be made, for example, to H. C. Brown et al., Acc. Chem. Res. 25, 17 (1992) and V. K. Singh, Synthesis, 605 (1992). In accordance with the invention the selective reducing agent is preferably a complex borohydride, such as LiBH₄, NaBH₄ or KBH₄, especially LiBH₄.

It has surprisingly been found that after the conversion of the dicarboxylic acid monoester, be it the free acid or its quaternary ammonium or phosphonium salt, into a metal salt in step (b) the required amount of the selective reducing agent in step (c), especially of LiBH₄, can be significantly reduced. Further, it has surprisingly been found that after the conversion of the dicarboxylic acid monoester, be it the free acid or its quaternary ammonium or phosphonium salt, into the lithium salt the yield of the reaction product, i.e. the lactone of the general formula (I), increases in spite of a reduction in the amount of selective reducing agent used, especially when LiBH₄ is used as the reducing agent.

The reduction of the dicarboxylic acid monoester can be effected in situ or also after its isolation. Depending on the reaction conditions which are chosen the dicarboxylic acid monoester of the general formula (VI) is present in protonated form of the general formula (VI a) or as a quaternary ammonium or phosphonium salt of the general formula (VI c), the quaternary ammonium or phosphonium group being derived from the catalyst of the general formula (V), or as a metal salt of the general formula (VI b) [formed in step (b)]. In a preferred embodiment of the process in accordance with the invention the free carboxylic acid function of the dicarboxylic acid monoester is converted into a metal salt [(step (b)] prior to the reaction with the selective reducing agent.

The reduction is preferably effected under an inert gas atmosphere, for example under nitrogen or argon, preferably in an inert, organic solvent such as an ether, for example dioxan or tetrahydrofuran or an ether of glycol or diethylene glycol, for example diglyme.

The reaction temperature is preferably 0° C. to 60° C., more preferably 30° C. to 45° C.

When the process in accordance with the invention embraces steps (a) and (c) but not step (b), i.e. not the intermediate metal salt formation, then preferably 2 to 3, more preferably 2.2 to 2.6, mol equivalents of the selective reducing agent are added based on the molar amount of the dicarboxylic acid monoester, be it the free acid or its quaternary ammonium or phosphonium salt. When the process in accordance with the invention embraces steps (a), (b) and (c), then preferably 1 to 2, more preferably 1.1 to 1.5, mol equivalents of the selective reducing agent are added. r

The selective reducing agent is preferably added in an inert ether as the solvent, tetrahydrofuran and dioxan being especially preferred. Mixtures of these inert ethers with inert aromatic hydrocarbons such as e.g. toluene are also suitable.

When the process in accordance with the invention embraces steps (a), (b) and (c), then in a preferred embodiment the reduction is effected with the addition of up to 2, preferably 0.5 to 1.5, mol equivalents of water. The water can either be added together with the dicarboxylic acid monoester metal salt or, however, dissolved in a solvent such as e.g. tetrahydrofuran or added separately.

In a preferred embodiment the process in accordance with the invention embraces steps (a) and (c), the selective reducing agent being a complex borohydride.

In a more preferred embodiment the process in accordance with the invention embracing steps (a) and (c) the catalyst in step (a) is a compound selected from the group consisting of tributylamine, tripentylamine, trihexylamine, trioctylamine, tridodecylamine and tributylphosphine and the reducing agent in step (c) is a metal borohydride.

In an especially preferred embodiment of the process in accordance with the invention embracing steps (a) and (c) the catalyst is tributylamine, trihexylamine or trioctylamine, the cyclic carboxylic acid anhydride is cis-1,3-dibenzyl-hexahydro-1H-furo[3,4-d]imidazole-2,4,6-trione, the chiral alcohol is (S)-1,1-diphenyl-1,2-propanediol and the selective reducing agent is lithium borohydride.

In a further especially preferred embodiment the process in accordance with the invention embraces steps (a), (b) and (c), the dicarboxylic acid monoester, be it the free acid or its quaternary ammonium or phosphonium salt, being converted in step (b) into the lithium salt.

The following Examples illustrate the process in accordance with the invention:

The diastereomeric purity and enantiomeric purity of the intermediates and final products can be determined, for example, by routine HPLC investigations. Such procedures will be known to a person skilled in the art.

### EXAMPLE 1

Synthesis of the Dicarboxylic Acid Monoester Using 10% Trioctylamine

0.177 g (0.5 mmol) of freshly distilled trioctylamine was added at 10° C. to a suspension of 1.68 g (5 mmol) of cis-1,3-dibenzyl-hexahydro-1H-furo[3,4-d]imidazole-2,4,6-trione and 1.14 g (5 mmol) of (S)-1,1-diphenyl-1,2-propanediol in 35 ml of toluene. The suspension was stirred at 20° C. for 18 hours, filtered and the residue was washed with 20 ml of toluene. The filter residue was dried.

There were obtained 2.52 g of the desired 5-[(S)-2-hydroxy-1-methyl-2,2-diphenyl-ethyl](4S,5R)-1,3-dibenzyl-2-oxo-4,5-imidazolidinedicarboxylate in a diastereomeric purity of 100% de and a total yield of 89% of theory.

### EXAMPLE 2

Synthesis of the Lithium Salt of the Dicarboxylic Acid Monoester Using 10% Trioctylamine for the Intermediate Synthesis of the Quaternary Ammonium Dicarboxylic Acid Monoester

0.12 ml (0.5 mmol) of freshly distilled trioctylamine was added at 10° C. to a suspension of 1.68 g (5 mmol) of cis-1,3-dibenzyl-hexahydro-1H-furo[3,4-d]imidazole-2,4,6-trione and 1.14 g (5 mmol) of (S)-1,1-diphenyl-1,2-propanediol in 35 ml of toluene. The suspension was stirred at 20° C. for 18 hours. It was treated with 5.7 ml of an aqueous 1N solution of lithium hydroxide. The suspension was filtered and the residue was washed with 25 ml of water. The filter residue was dried.

There were obtained 2.53 g of the desired lithium 5-[(S)-2-hydroxy-1-methyl-2,2-diphenylethyl](4S,5R)-1,3-dibenzyl-2-oxo-4,5-imidazolidinedicarboxylate in a diastereomeric purity of 98.1% de.

### EXAMPLE 3

Synthesis of the Lithium Salt of the Dicarboxylic Acid Monoester Using an Equimolar Amount of Tributyl Amine for the Intermediate Synthesis of the Quaternary Ammonium Dicarboxylic Acid Monoester

11.93 ml (50 mmol) of freshly distilled tributylamine were added dropwise at −5° C. to a suspension of 16.82 g (50 mmol) of cis-1,3-dibenzyl-hexahydro-1H-furo[3,4-d]imidazole-2,4,6-trione and 11.64 g (51 mmol) of (S)-1,1-diphenyl-1,2-propanediol in 100 ml of toluene. The suspension was stirred at 0° C. for 1 hour and subsequently at room temperature for 1 hour. The solution of the formed tributylammonium 5-[(S)-2-hydroxy-1-methyl-2,2-diphenylethyl](4S,5R)-1,3-dibenzyl-2-oxo-4,5-imidazolidinedicarboxylate was treated with 57 ml (57 mmol) of an aqueous 1N solution of lithium hydroxide. The suspension was stirred for 4 hours, filtered and the residue was washed with 50 ml of water. The filter residue was dried in a vacuum.

There were obtained 27.5 g of the desired lithium 5-[(S)-2-hydroxy-1-methyl-2,2-diphenylethyl](4S,5R)-1,3-dibenzyl-2-oxo-4,5-imidazolidinedicarboxylate in a diastereomeric purity of 98.7% de (determined by HPLC) and a total yield of 96% of theory.

### EXAMPLE 4

Reduction of the Lithium Salt of the Dicarboxylic Acid Monoester 27.4 g (48 mmol) of lithium 5-[(S)-2-hydroxy-1-methyl-2,2-diphenylethyl](4S,5R)-1,3-dibenzyl-2-oxo-4,5-imidazolidinedicarboxylate were added to a solution of 1.35 g (62 mmol) of lithium borohydride in 50 ml of tetrahydrofuran. Within 4 hours the mixture was treated at 20° C. with 5 ml (50 mmol) of a 10M solution of water in tetrahydrofuran. The suspension was stirred at 20° C. for a further 2 hours and at 40° C. for 2 hours and treated with 100 ml of water and 100 ml of toluene. After stirring for 30 minutes the aqueous phase was separated and extracted three times with 100 ml of toluene each time. The organic phases were washed twice with 50 ml of water each time, combined and concentrated.

There were obtained 10.2 g of (S)-1,1-diphenyl-1,2-propanediol with a total yield of 89% of theory.

The aqueous phases were combined and partially concentrated in a vacuum. After acidification with 40 ml of 4M hydrochloric acid (160 mmol) the suspension was stirred at 90° C. for 1 hour. The precipitated d-lactone was filtered off and washed with 50 ml of water. The filter cake was dried in a vacuum for 2 hours. There were obtained 15.12 g of the d-lactone, i.e. (3aS,6aR)-1,3-dibenzyl-dihydro-1H-furo[3,4-d]imidazol-2,4(3H,3aH)-dione, as a crude product in an enantiomeric purity of 98.5% ee.

### EXAMPLE 5

Comparison of Various Catalysts

Analogously to the procedure described in Example 1, cis-1,3-dibenzyl-hexahydro-1H-furo[3,4-d]imidazole-2,4,6-trione was reacted with (S)-1,1-diphenyl-1,2-propanediol in toluene using different catalysts which in some cases were employed in different amounts:

The results thereby obtained are compiled in the following Table. In addition to the diastereomeric purity of the appropriate 5-[(S)-2-hydroxy-1-methyl-2,2-diphenylethyl](4S,5R)-1,3-dibenzyl-2-oxo-4,5-imidazolidinedicarboxylate obtained in [% de] there are also given the mol equivalents of the catalyst employed with respect to the amount of the cis-1,3-dibenzyl-hexahydro-1H-furo[3,4-d]imidazole-2,4,6-trione employed:

TABLE 1

| | Mol | |
|---|---|---|
| QR⁸R⁹R¹⁰ | equivalents | % de |
| N(CH₃)₃ | 1.0 | 93.7 |
| N(CH₂CH₃)₃ | 1.0 | 93.0 |
| Diazabicyclooctane | 1.0 | 96.6 |
| N[(CH₂)₂CH₃]₃ | 1.0 | 96.3 |
| N[(CH₂)₃CH₃]₃ | 1.0 | 97.6 |
| N[(CH₂)₃CH₃]₃ | 0.1 | 97.3 |
| N[(CH₂)₅CH₃]₃ | 1.0 | 97.8 |
| N[(CH₂)₇CH₃]₃ | 1.0 | 98.0 |
| N[(CH₂)₇CH₃]₃ | 0.05 | 96.6 |
| N[(CH₂)₁₁CH₃]₃ | 1.0 | 98.2 |
| P[(CH₂)₃CH₃]₃ | 1.0 | 98.1 |

### EXAMPLE 6

Comparison of the Diastereomeric Purity of the Dicarboxylic Acid Monoester Immediately After the Esterification and After Isolation of the Tributylammonium Salt

Tributylammonium 5-[(S)-2-hydroxy-1-methyl-2,2-diphenylethyl](4S,5R)-1,3-dibenzyl-2-oxo-4,5-imidazolidinedicarboxylate was prepared analogously to the procedure described in Example 3, but conversion into the lithium salt was not carried out. The diastereomeric purity of the tributylammonium salt immediately after the reaction and after isolation of the salt are contrasted in the following Table:

TABLE 2

| | | Mol | |
|---|---|---|---|
| | QR⁸R⁹R¹⁰ | equivalents | % de |
| Before isolation | N[(CH₂)₃CH₃]₃ | 1.0 | 97.6 |
| After isolation | N[(CH₂)₃CH₃]₃ | 1.0 | 99.8 |

### EXAMPLE 7

Isolation of the Dicarboxylic Acid Monoester as the Alkali Metal Salt

Tributylammonium 5-[(S)-2-hydroxy-1-methyl-2,2-diphenylethyl](4S,5R)-1,3-dibenzyl-2-oxo-4,5-imidazolidinedicarboxylate was converted into different alkali metal salts analogously to the procedure described in Example 3 and subsequently reduced with lithium borohydride analogously to the procedure described in Example 4. The results are contrasted in the following Table, and the names of the salts are given subsequently:

TABLE 3

| | | Consumption |
|---|---|---|
| Alkali | Yield | LiBH₄ [mol |
| metal | [mol %] | equivalents] |
| Li | 96 | 1.3 |
| Na | 91 | 1.5 |
| K | 90 | 1.6 |

Lithium 5-[(S)-2-hydroxy-1-methyl-2,2-diphenylethyl](4S,5R)-1,3-dibenzyl-2-oxo-4,5-imidazolidinedicarboxylate
Sodium 5-[(S)-2-hydroxy-1-methyl-2,2-diphenylethyl](4S,5R)-1,3-dibenzyl-2-oxo-4,5-imidazolidinedicarboxylate
Potassium 5-[(S)-2-hydroxy-1-methyl-2,2-diphenylethyl](4S,5R)-1,3-dibenzyl-2-oxo-4,5-imidazolidinedicarboxylate

1. A process for the enantioselective synthesis of a chiral lactone of the general formula (I) 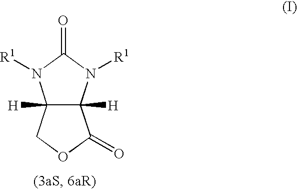
wherein R¹ is benzyl, α-phenylethyl, allyl, 1-furyl, 2-furyl, 1-thienyl, 2-thienyl or p-methoxybenzyl,
from a cyclic carboxylic acid anhydride of the general formula (II) 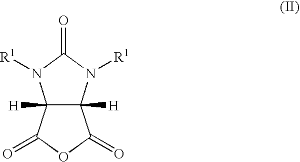
wherein R¹ is as previously defined,
with the aid of a chiral alcohol of the general formula (III) 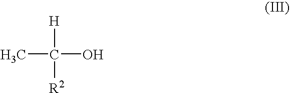
wherein R² is a residue of the general formulae (IV a-f) 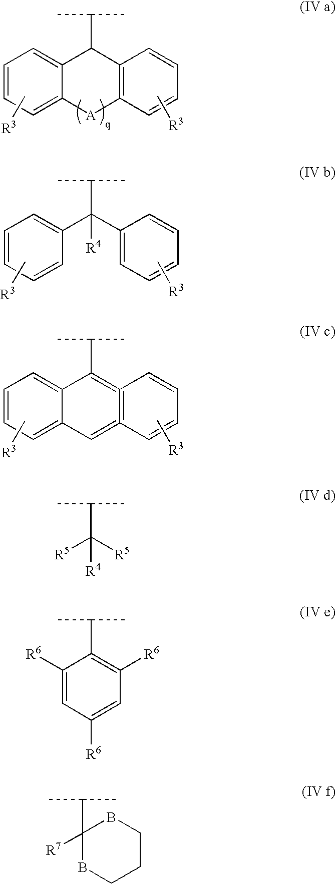
wherein
R³ is hydrogen, fluorine, chlorine, bromine, iodine, C₁-C₆-alkyl or C₁-C₆-alkoxy,
R⁴ is hydrogen, hydroxyl, C₁-C₆-alkyl, C₁-C₆-alkoxy or phenyl,
R⁵ is C₃-C₇-cycloalkyl, phenyl optionally substituted with chlorine or methyl, pyridyl, pyrrolyl, thienyl or furyl,
R⁶ is hydrogen or C₁-C₆-alkyl,
R⁷ is C₁-C₆-alkyl or phenyl,
A is sulphur or a methylene group, q being the integer 1 when A is sulphur or q being the integer 1 or 2 when A is a methylene group, and
B is sulphur, —SO2- or a methylene group, including the step
(a) bringing together the cyclic carboxylic acid anhydride with the chiral alcohol for esterification in the presence of a catalyst of the general formula (V) 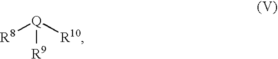
wherein
Q is nitrogen or phosphorus and
R⁸, R⁹ and R¹⁰ are each independently
(i) C₂-C₁₈₋-alkyl in which optionally up to two methylene groups can be replaced by oxygen, or
(ii) phenyl-C₁-C₄-alkyl in which optionally one methylene group can be replaced by oxygen, or
(iii) phenyl,
with the proviso that
when one of the residues R⁸, R⁹ and R¹⁰ is phenyl the other two residues are not phenyl
and
when R⁸, R⁹ and R¹⁰ are each C₂-C₁₈-alkyl at least one of the three substituents R⁸, R⁹ or R¹⁰ comprises at least 3 carbon atoms.

2. A process according to claim 1, characterized in that R⁸, R⁹ and R¹⁰ are each independently C₃-C₁₂-alkyl.

3. A process according to claim 2, characterized in that R⁸, R⁹ and R¹⁰ are identical.

4. A process according to claim 1, characterized in that Q is nitrogen.

5. A process according to claim 1, characterized in that R¹ is benzyl.

6. A process according to claim 1, characterized in that R²is a residue of the general formula (IV d).

7. A process according to claim 6, characterized in that R⁴ is hydrogen or hydroxyl and R⁵ is phenyl optionally substituted with chlorine or methyl, or is thienyl or 2-furyl.

8. A process according to claim 1, characterized in that the chiral alcohol is (S)-1,1-diphenyl-1,2-propanediol.

9. A process according to claim 1, characterized in that the process includes in addition to step (a) the step
(b) conversion of this form of the dicarboxylic acid monoester obtained in step (a) into a metal salt.

10. A process according to claim 9, characterized in that the metal salt is an alkali metal salt.

11. A process according to claim 10, characterized in that the alkali metal salt is the lithium salt.

12. A process according to claim 1, characterized in that the process includes the step
(c) bringing together the dicarboxylic acid monoester obtained in step (a) or the dicarboxylic acid monoester metal salt obtained in step (b) with a reducing agent which is selective for ester groups.

13. A process according to claim 12, characterized in that the selective reducing agent is a complex borohydride.

14. A process according to claim 13, characterized in that the selective reducing agent is lithium borohydride.

15. A process according to claim 12, characterized in that the reduction is carried out with the addition of 0.5 to 1.5 mol equivalents of water.

16. A process according to claim 12, characterized in that step (c) is carried out directly after step (a) and the selective reducing agent is a complex borohydride.

17. A process according to claim 16, characterized in that the selective reducing agent is lithium borohydride.
